(19) United States
(12) Patent Application Publication
(10) Pub. No.: US 2013/0225611 A1
(43) Pub. Date: Aug. 29, 2013

(54) Compositions and Methods for Reducing Cancer Cell Proliferation and Modulating Importin-Beta Function

(71) Applicant: The Regents of the University of California, (US)

(72) Inventors: Karsten Weis, Berkeley, CA (US); Rebecca Heald, Berkeley, CA (US); Jonathan F. Soderholm, Richmond, CA (US); Stephen L. Bird, Berkeley, CA (US); Petr Kalab, Berkeley, CA (US)

(73) Assignee: THE REGENTS OF THE UNIVERSITY OF CALIFORNIA, Oakland, CA (US)

(21) Appl. No.: 13/705,538

(22) Filed: Dec. 5, 2012

Related U.S. Application Data
(60) Provisional application No. 61/568,861, filed on Dec. 9, 2011.

(51) Int. Cl. C07D 239/95

(52) U.S. Cl. CPC C07D 239/95
USPC 514/266.2; 435/375

(57) ABSTRACT

The present disclosure provides methods of reducing proliferation of cancer cells. The methods include contacting the cancer cells with a 2,4-diaminoquinazoline compound. Also provided are methods of modulating importin-beta function (e.g., importin-beta-mediated nuclear import) in eukaryotic cells. The methods include contacting the eukaryotic cells with a 2,4-diaminoquinazoline compound. Pharmaceutical formulations that include a 2,4-diaminoquinazoline compound are also provided.

### CROSS-REFERENCE

This application claims the benefit of U.S. Provisional Patent Application No. 61/568,861, filed Dec. 9, 2011, which application is incorporated herein by reference in its entirety.

### STATEMENT REGARDING FEDERALLY SPONSORED RESEARCH

This invention was made with government support under Grant Nos. GM065232 and NS053592 awarded by the National Institutes of Health. The government has certain rights in the invention.

### BACKGROUND

Importin-β transport receptors, which comprise at least 22 members in vertebrates, bind to cargo molecules and mediate their import or export through nuclear pores. Directionality of transport depends on the nature of the receptor as well as the asymmetric distribution of nucleotide states of the small GTPase Ran, which is GTP-bound in the nucleus due to the chromatin interaction of its guanine exchange factor (GEF) RCC1, and GDP-bound in the cytoplasm where its GTPase activating protein, RanGAP, is localized. The founding member of this family, importin-β, together with its partner importin-α, recognize nuclear localization signal (NLS)-containing cargo molecules and transport them into the nucleus where RanGTP binds directly to importin-β, causing a conformational change that releases importin-β and NLS-cargoes. In addition to its vital interphase functions, importin-β and Ran are also important regulators during mitosis, contributing to chromatin-mediated spindle assembly. During mitosis, importin-β has an inhibitory function towards NLS-containing spindle assembly factors, binding them in the cytoplasm and impairing their microtubule-stabilizing or organizing activities. However, RanGTP remains enriched around condensed mitotic chromosomes in mitosis and generates a gradient of released cargoes that triggers spindle assembly. The importin-β/RanGTP pathway has also been implicated in a variety of other cellular processes including postmitotic nuclear envelope assembly, nuclear pore complex assembly, protein ubiquitylation, and primary cilium formation.

### LITERATURE

U.S. Pat. No. 5,444,062; Soderholm et al. (2011) ACS Chem. Biol. 6:700-708; Hintersteiner et al. (2010) ACS Chem. Biol. 5:967-979; Schornagel et al. (1984) Biochem. Pharm. 33(20):3251-3255.

### SUMMARY

The present disclosure provides methods of reducing proliferation of cancer cells. The methods include contacting the cancer cells with a 2,4-diaminoquinazoline compound. Also provided are methods of modulating importin-beta function (e.g., importin-beta-mediated nuclear import) in eukaryotic cells. The methods include contacting the eukaryotic cells with a 2,4-diaminoquinazoline compound. Pharmaceutical formulations that include a 2,4-diaminoquinazoline compound are also provided.

### BRIEF DESCRIPTION OF THE DRAWINGS

FIGS. 1A-E depict various aspects of Ran-GTP. FIG. 1A is a schematic of the fusion proteins that bind and undergo FRET in the presence of Ran-GTP but not Ran-GDP. FIG. 1B shows the fluorescence emission of the FRET pair detected between 460 nm and 550 nm following excitation at 435 nm, showing strong emission of CFP (475 nm) in the presence of GDP (red curve) that decreases in the presence of GTP (blue curve) concomitant with an increase at the emission wavelength of YFP (525 nm), indicative of FRET. FIG. 1C summarizes a screen to identify modulators of FRET between CFP-Ran and YFP-importin-β. Of 137,284 small molecules screened in duplicate using the FRET-based assay, 141 putative hits were subjected to three secondary screens designed to eliminate false positives. Of the 10 compounds remaining after the secondary screens, a compound was identified that reproducibly diminished the FRET signal generated by CFP-Ran and YFP-importin-β in the original assay (FIG. 1D). FIG. 1E shows the structure of importazole, a 2,4-diaminoquinazoline.

FIGS. 2A-C depict the effect of importazole on importin-β. FIG. 2A is a graph showing negative first derivatives of melting curves of 2 μM importin-β in the presence of 50 μM importazole or DMSO where the minima indicate the melting temperature. Melting curves show the results from six experiments conducted in quadruplicate using the Applied Biosystems 7500 qPCR machine. FIG. 2B is a graph showing negative first derivatives of melting curves of 2 μM RanQ69L in the presence of 50 μM importazole or DMSO as control where the minima indicate the melting temperature. FIG. 2C is a bar graph that shows the mean changes in melting temperature of 2 μM importin-β, RanQ69L, transportin and CRM1 in the presence of 50 μM importazole. Error bars indicate standard error; asterisks denote statistical significance (p<0.01).

FIGS. 3A-C depict the effect of importazole on nuclear localization. FIG. 3A shows the results of a fluorescence microscopy assay where NLS-GFP (importin-β import substrate) was added with Xenopus egg extract to permeabilized HeLa cells and assayed by fluorescence microscopy for nuclear import in the presence of DMSO or 100 μM importazole. FIG. 3B shows NLS-GFP accumulation at the nuclear rim in the presence of importazole. FIG. 3C shows M9-YFP (transportin import substrate) in the same assay. In all figures, scale bar=10 μm.

FIGS. 4A-C depict nuclear localization of NFAT. FIG. 4A is a schematic showing that the GFP-tagged, NLS-containing transcription factor NFAT enters the nucleus upon treatment with the ionophore ionomycin in a RanGTP- and importin-β-dependent manner. FIG. 4B provides fluorescent microscopic images of HEK 293 cells stably expressing GFP-NFAT and treated with DMSO or 40 μM importazole for 1 hour prior to a 30 min treatment with ionomycin to induce nuclear import. Importazole was washed out and after 1 hour prior to ionomycin re-treatment. FIG. 4C shows the results quantified as the percentage of cells with nuclear NFAT-GFP. N=3, 100 or more cells counted under each condition. Bars represent standard error.

FIGS. 5A-C depict the effect of ionomycin on nuclear localization. FIG. 5A is a schematic illustrating that upon removal of the ionophore ionomycin, GFP-NFAT exits the nucleus in a RanGTP- and Crm1-dependent manner. FIG. 5B provides fluorescent microscopic images of cells treated with ionomycin to induce nuclear import of NFAT-GFP, then washed and treated with DMSO, importazole, leptomycin B, or importazole+leptomycin B for 1 hour. FIG. 5C shows the results quantified as the percentage of cells with nuclear NFAT-GFP. N=3, 100 or more cells counted under each condition. Bars represent standard error.

FIGS. 6A-E depict the effect of importazole on spindle assembly. FIG. 6A shows fluorescent microscopic images of spindle assembly reactions containing X-rhodamine labeled tubulin in the presence of DMSO, 100 μM importazole, or a truncated form of importin-β that is unable to bind to RanGTP. Microtubules are red and DNA is blue. FIG. 6B shows the quantification of the percentage of normal spindle structures. N=3, 100 structures counted under each condition. Bars represent standard error. FIG. 6C shows aster assembly induced by addition of 5% DMSO to extracts containing X-rhodamine labeled tubulin in the presence of DMSO or importazole. FIG. 6D is a bar graph quantifying the number of asters per field. 10 fields were counted under each condition. FIG. 6E is a photograph of an SDS PAGE gel showing the results of a DMSO-induced pure tubulin polymerization assay. Reactions were supplemented with additional DMSO, importazole, or nocodazole, and microtubules pelleted through a sucrose cushion and samples from the pellet (P) and supernatant (S) analyzed by SDS PAGE.

FIG. 7 provides images indicating that importazole disrupts mitotic cargo release as monitored by the FRET probe Rango. Donor fluorescence (top panels) and pseudo-colored FLIM images (bottom panels) of mitotic HeLa cells expressing the Rango-3 FRET sensor. Rango-3 displays a greater fluorescence lifetime around the chromosomes of cells treated with importazole compared to that of cells treated with DMSO, resulting from importazole's disruption of sensor release from importin-β. N=3, 30 cells counted for each condition.

FIGS. 8A-C depict the effect of importazole on spindle positioning. FIG. 8A shows fluorescent microscopic images of asynchronously growing cultures were treated with DMSO or importazole for 1 hour prior to fixation and staining for DNA (blue) and tubulin (red). Defects including chromosome congression (white arrowheads point to misaligned chromosomes) and spindle positioning upon importazole treatment are apparent. Dashed white lines indicate cell boundaries. FIG. 8B shows quantification of spindle defects in cells treated with DMSO, 20 importazole, or 40 μM importazole. N=5. In each case, 100 metaphase cells were counted and the fraction of those displaying defects was scored. FIG. 8C shows time-lapse fluorescent microscopic images of metaphase HeLa cells treated with 50 μM importazole. Frames were captured every 3 minutes. FIG. 8D is a bar graph showing the spindle area of asychnronous HeLa cells treated with 0 to 40 μM importazole for 1 hour prior to fixation. The size of the spindle in mitotic cells was measured. N=4, 100 metaphase spindles were measured per condition. Bars represent standard error.

### DEFINITIONS

The terms “subject,” “individual,” and “patient” are used interchangeably herein to a member or members of any mammalian or non-mammalian species that may have a need for the pharmaceutical methods, compositions and treatments described herein. Subjects and patients thus include, without limitation, primate (including humans and non-human primates), canine, feline, ungulate (e.g., equine, bovine, swine (e.g., pig)), avian, and other subjects. Humans and non-human mammals having commercial importance (e.g., livestock and domesticated animals) are of particular interest.

“Mammal” refers to a member or members of any mammalian species, and includes, by way of example, canines; felines; equines; bovines; ovines; rodentia, etc. and primates, e.g., humans. Non-human animal models, particularly mammals, e.g. a non-human primate, a murine (e.g., a mouse, a rat), lagomorpha, etc. may be used for experimental investigations.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. Cancerous cells can be benign or malignant.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “isolated compound” means a compound which has been substantially separated from, or enriched relative to, other compounds with which it occurs in nature. Isolated compounds are at least about 80%, at least about 90% pure, at least about 98% pure, or at least about 99% pure, by weight. The present disclosure is meant to comprehend diastereomers as well as their racemic and resolved, enantiomerically pure forms and pharmaceutically acceptable salts thereof.

A “therapeutically effective amount” or “efficacious amount” means the amount of a compound that, when administered to a mammal or other subject for treating a disease or condition, is sufficient, in combination with another agent, or alone in one or more doses, to effect such treatment for the disease or condition. The “therapeutically effective amount” will vary depending on the compound, the disease and its severity and the age, weight, etc., of the subject to be treated.

A “pro-drug” means any compound that releases an active parent drug according to one or more of the generic formulas shown below in vivo when such pro-drug is administered to a mammalian subject or mammalian cells. Pro-drugs of a compound of one or more of the generic formulas shown below are prepared by modifying functional groups present in the compound of the generic formula in such a way that the modifications may be cleaved in vivo to release the parent compound. Pro-drugs include compounds of one or more of the generic formulas shown below wherein a hydroxy, amino, or sulfhydryl group in one or more of the generic formulas shown below is bonded to any group that may be cleaved in vivo to regenerate the free hydroxyl, amino, or sulfhydryl group, respectively. Examples of pro-drugs include, but are not limited to esters (e.g., acetate, formate, and benzoate derivatives), carbamates (e.g., N,N-dimethylaminocarbonyl) of hydroxy functional groups in compounds of one or more of the generic formulas shown below, and the like.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

“In combination with,” or “co-administration,” as used herein, in the context of administering a first compound and at least a second compound, refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The term “unit dosage form,” as used herein, refers to physically discrete units suitable as unitary dosages for human and animal subjects, each unit containing a predetermined quantity of a compound, calculated in an amount sufficient to produce the desired effect in association with a pharmaceutically acceptable diluent, carrier or vehicle. The specifications for a dosage form may depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and is free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal and the like. In some embodiments the composition is suitable for administration by a transdermal route, using a penetration enhancer other than dimethylsulfoxide (DMSO). In other embodiments, the pharmaceutical compositions are suitable for administration by a route other than transdermal administration. A pharmaceutical composition will in some embodiments include a compound (e.g., a 2,4-diaminoquinazoline compound such as a compound having a structure of Formula I, Formula Ia, Formula Ib, importazole, etc.) and a pharmaceutically acceptable excipient. In some embodiments, a pharmaceutically acceptable excipient is other than DMSO.

As used herein, “pharmaceutically acceptable derivatives” of a compound of the present disclosure include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

A “pharmaceutically acceptable ester” of a subject compound means an ester that is pharmaceutically acceptable and that possesses the desired pharmacological activity of the parent compound, and includes, but is not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

A “pharmaceutically acceptable enol ether” of a subject compound means an enol ether that is pharmaceutically acceptable and that possesses the desired pharmacological activity of the parent compound, and includes, but is not limited to, derivatives of formula C═C(OR) where R is hydrogen, alkyl, alkenyl, alkynyl, aryl, heteroaryl, aralkyl, heteroaralkyl, cycloalkyl or heterocyclyl.

A “pharmaceutically acceptable solvate or hydrate” of a subject compound means a solvate or hydrate complex that is pharmaceutically acceptable and that possesses the desired pharmacological activity of the parent compound, and includes, but is not limited to, complexes of a subject compound with one or more solvent or water molecules, or 1 to about 100, or 1 to about 10, or one to about 2, 3 or 4, solvent or water molecules.

The term “organic group” and “organic radical” as used herein means any carbon-containing group, including hydrocarbon groups that are classified as an aliphatic group, cyclic group, aromatic group, functionalized derivatives thereof and/or various combinations thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different. The term “dihaloalkyl” refers to an alkyl group as described above that is substituted by two halo groups, which may be the same or different. The term “trihaloalkyl” refers to an alkyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N—(C₁ to C₆ alkyl)carboxamide, protected N—(C₁ to C₆ alkyl)carboxamide, N,N-di(C₁ to C₆ alkyl)carboxamide, trifluoromethyl, N—((C₁ to C₆ alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to C₄ alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(alkyl)” denotes an alkyl group as defined above, substituted at any position by a heteroaryl group, as above defined.

“Optional” or “optionally” means that the subsequently described event, circumstance, feature, or element may, but need not, occur, and that the description includes instances where the event or circumstance occurs and instances in which it does not. For example, “heterocyclo group optionally mono- or di-substituted with an alkyl group” means that the alkyl may, but need not, be present, and the description includes situations where the heterocyclo group is mono- or disubstituted with an alkyl group and situations where the heterocyclo group is not substituted with the alkyl group.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof A mixture containing equal proportions of the enantiomers is called a “racemic mixture.”

A compound may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see, e.g., the discussion in Chapter 4 of “Advanced Organic Chemistry”, 4th edition J. March, John Wiley and Sons, New York, 1992).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a 2,4-diaminoquinazoline compound” includes a plurality of such compounds and reference to “the cancer cell” includes reference to one or more such cancer cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of reducing proliferation of cancer cells. The methods include contacting the cancer cells with a 2,4-diaminoquinazoline compound. Also provided are methods of modulating importin-beta-mediated nuclear import in eukaryotic cells. The methods include contacting the eukaryotic cells with a 2,4-diaminoquinazoline compound. Pharmaceutical formulations that include a 2,4-diaminoquinazoline compound are also provided.

### Compounds

The present disclosure provides compounds that find a variety of uses, such as to reduce proliferation of cancer cells and/or to modulate importin-β-mediated nuclear import in a eukaryotic cell. These compounds can be incorporated into a variety of formulations, e.g., for therapeutic administration by a variety of routes. For example, the compounds disclosed herein can be formulated into pharmaceutical compositions by combination with appropriate, pharmaceutically acceptable carriers, diluents, excipients and/or adjuvants.

In certain embodiments, the subject compounds include a substituent that contributes to optical isomerism and/or stereo isomerism of a compound. Salts, solvates, hydrates, and prodrug forms of a compound are also of interest. All such forms are embraced by the present invention. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

The following are examples of compounds of the present disclosure.

### 2,4-diaminoquinazolines and Derivatives Thereof

In certain embodiments, a subject compound is a 2,4-diaminoquinazoline or a 2,4-diaminoquinaoline derivative. In some embodiments, the compound is a compound of Formula I, as shown below.

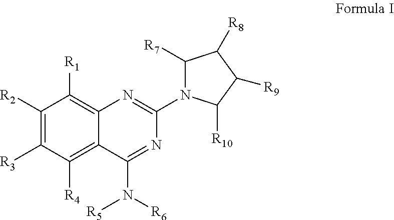

where R1, R2, R3, R4, R5, R6, R7, R8, R9 and R10 are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;

or a pro-drug, a pharmaceutically acceptable salt, an analog, or a derivative thereof.

In certain aspects, the compound has the structure of Formula Ia, as shown below.

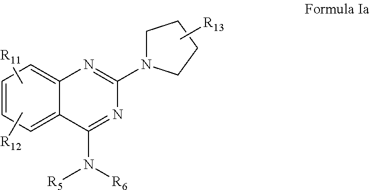

where R₅ and R₆ are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and

where R₁₁, R₁₂ and R₁₃ are each attached to their respective rings at any available position on the ring and are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;

or a pro-drug, a pharmaceutically acceptable salt, an analog, or a derivative thereof.

The compound may have the structure of Formula Ib, as shown below.

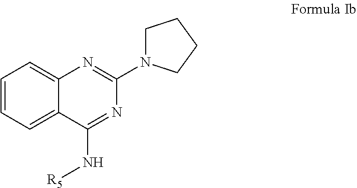

where R5 is selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;

or a pro-drug, a pharmaceutically acceptable salt, an analog, or a derivative thereof.

In some instances, R₅ is a substituted or unsubstituted alkyl group. For example, in some embodiments, R₅ is:

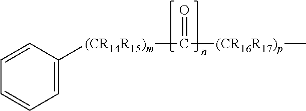

where R₁₄, R₁₅, R₁₆ and R₁₇ are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and

m, n and p are each independently an integer from 0 to 6.

In certain aspects, R₅ is:

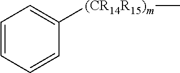

where R₁₄ and R₁₅ are each independently selected from H, a halogen (e.g., F, Br, Cl, or I), a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and

m is an integer from 1 to 6.

According to one embodiment, the compound is (1-phenyl-ethyl)-(2-pyrrolidin-1-yl-quinazolin-4-yl)-amine (also known as importazole, IPZ) and has the following structure:

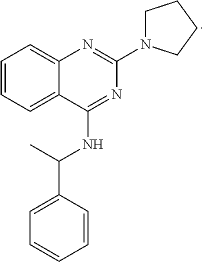 [SMILES: CC(Nc1nc(N2CCCC2)nc2ccccc12)c1ccccc1]

### Methods of Modulating Importin-Beta Function

The present disclosure provides methods of modulating importin-β function. In certain aspects, the present disclosure provides methods of modulating importin-β-mediated nuclear import in a eukaryotic cell, the methods including contacting a eukaryotic cell of interest with one or more compounds described herein, e.g., a 2,4-diaminoquinazoline compound such as a compound of Formula I, Formula Ia, and Formula Ib. In certain aspects, the methods include contacting a eukaryotic cell of interest with a compound that is (1-phenyl-ethyl)-(2-pyrrolidin-1-yl-quinazolin-4-yl)-amine (also known as importazole, IPZ).

In certain embodiments, the subject methods modulate importin-β-mediated nuclear import by affecting the interaction of importin-β with RanGTP. For example, contacting the eukaryotic cell with a compound of the present disclosure (e.g., importazole or any suitable compound of Formula I, Formula Ia, or Formula Ib) may disrupt the interaction of importin-β with RanGTP, thereby disrupting importin-β/RanGTP-mediated nuclear import.

In certain embodiments, contacting eukaryotic cells of interest with a subject compound (e.g., importazole) is carried out while the cells are in interphase and results in a reduction in nuclear import in the contacted eukaryotic cells as compared to control cells not contacted with the subject compound. The subject methods may result in a reduction of nuclear import of a protein (e.g., a protein having a nuclear localization signal (NLS)) by 10%, 20%, 30%, 40%, 50%, 60%, 70%, 80%, 90% or more as compared to control cells not contacted with the subject compound.

In other aspects, the subject methods result in a disruption of mitosis in the contacted eukaryotic cells. For example, contacting eukaryotic cells of interest with a subject compound (e.g., importazole or any other 2,4-diaminoquinazoline compound such as a compound having a structure of Formula I, Formula Ia, or Formula Ib) may result in increased blockage of mitotic spindle assembly in metaphase-arrested eukaryotic cells. In certain aspects, blockage of mitotic spindle assembly may be increased by 10%, 20%, 30%, 40%, 50%, 60%, 70%, 80%, 90% or more as compared to control metaphase-arrested eukaryotic cells not contacted with the subject compound. The subject methods may result in complete or increased blockage of mitotic spindle assembly without affecting pure microtubules.

In certain embodiments, the subject methods result in impaired importin-β mitotic cargo release. According to certain aspects of the present disclosure, contacting eukaryotic cells of interest with a subject compound (e.g., importazole or any other 2,4-diaminoquinazoline compound such as a compound having a structure of Formula I, Formula Ia, or Formula Ib) may result in reduced importin-β mitotic cargo release. For example, importin-β mitotic cargo release may be reduced by 10%, 20%, 30%, 40%, 50%, 60%, 70%, 80%, 90% or more as compared to control eukaryotic cells not contacted with the subject compound.

In certain aspects, the subject methods modulate importin-β function, thereby effecting a variety of cellular processes in addition to those mentioned above, such as postmitotic nuclear envelope assembly, nuclear pore complex assembly, protein ubiquitylation, and primary cilium formation.

The subject methods are useful in a variety of applications, both in the clinical and research settings. For example, the methods of modulating importin-β function using the compounds of the present disclosure may be used to reduce cell proliferation (e.g., cancer cell proliferation) in a subject afflicted with a disease resulting from abnormal cell proliferation as described in more detail below. In addition, the subject methods find use as research tools for studying fundamental biological processes such as nuclear import, mitotic spindle assembly, mitotic cargo release, postmitotic nuclear envelope assembly, nuclear pore complex assembly, protein ubiquitylation, and primary cilium formation.

### Methods of Reducing Proliferation of Cancer Cells

Also provided by the present disclosure are methods of reducing proliferation of cancer cells, the methods including contacting cancer cells of interest with one or more compounds described herein, e.g., a 2,4-diaminoquinazoline compound such as a compound of Formula I, Formula Ia, and Formula Ib. In certain aspects, the methods include contacting a cancer cell with a compound that is (1-phenyl-ethyl)-(2-pyrrolidin-1-yl-quinazolin-4-yl)-amine (also known as importazole, IPZ).

According to one embodiment, a subject method involves contacting cancer cells of interest in vitro, in vivo, or ex vivo with an effective amount of a subject compound (e.g., a 2,4-diaminoquinazoline compound such as a compound having a structure of Formula I, Formula Ia, Formula Ib, importazole, etc.), wherein the contacting reduces proliferation of the cancer cells by at least about 10%. By “effective amount” is meant an amount of the anti-proliferative compound that is sufficient to inhibit the growth of the target cell population to a desired level. In certain aspects, the subject compound reduces proliferation of the cancer cells by at least 10%, at least 15%, at least 20%, at least 25%, at least 40%, at least 50%, at least 75%, at least 2-fold, at least 2.5-fold, at least 5-fold, at least 10-fold, or more than 10-fold, compared to the proliferation of similar cancer cells not contacted with the subject compound.

In certain embodiments, a subject compound (e.g., importazole and/or any other 2,4-diaminoquinazoline compound described herein) reduce proliferation of cancer cells, which reduction in proliferation may be measured, for example, by a cell proliferation assay that measures the absorbance or fluorescence of a marker of the density of cells in culture, relative to a control, i.e., a subject compound show signals that are reduced by at least 10%, such as at least 20%, at least 30%, at least 40%, at least 50%, at least 60%, at least 70%, at least 80%, at least 90%, at least 95%, or even more, relative to a control signal. For example a compound of the present disclosure may inhibit growth of cancer cells with an IC₅₀ value of 50 μM or less, 40 μM or less, 30 μM or less, 20 μM or less, 10 μM or less, 5 μM or less, 3 μM or less, 1 μM or less, 300 nM or less, 100 nM or less, 30 nM or less, 10 nM or less, 3 nM or less, or 1 nM or less. Cancer cell growth inhibition can be determined using a cell proliferation assay.

In practicing methods of the present disclosure, the cells of interest may be contacted with the effective amount of the anti-proliferative compound (e.g., a 2,4-diaminoquinazoline compound such as a compound having a structure of Formula I, Formula Ia, Formula Ib, importazole, etc.) in an in vitro or ex vivo culture system, or in vivo. For example, an anti-proliferative compound may be contacted to primary cells grown under standard tissue culture conditions, or alternatively, to cells that are part of a whole animal (e.g., administered to a subject, such as a human or other mammal). As such, the target cell or collection of cells may vary, where the collection of cells may be cultured cells, a whole animal or portion thereof, e.g., tissue, organ, etc. As such, the target cell(s) may be a host animal or portion thereof.

In the subject methods, the anti-proliferative compound (e.g., a 2,4-diaminoquinazoline compound such as a compound having a structure of Formula I, Formula Ia, Formula Ib, importazole, etc.) may be contacted with the target cells using any convenient protocol that results in the desired level of anti-proliferative activity. Thus, the anti-proliferative compound can be incorporated into a variety of pharmaceutical compositions for therapeutic administration, e.g., as described below. For example, the anti-proliferative compound can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols, such as described above. As such, administration of the anti-proliferative compound can be achieved in various ways, including oral, buccal, rectal, parenteral, intraperitoneal, intradermal, transdermal, intracheal, etc., administration.

The subject methods find use in the treatment of a variety of different conditions, e.g., in which inhibition of the growth of cancer cells in a host is desired. By treatment is meant that at least an amelioration of the symptoms associated with the condition afflicting the host is achieved, where amelioration is used in a broad sense to refer to at least a reduction in the magnitude of a parameter, e.g. symptom (such as tumour growth or cell population growth), associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In one aspect of the present disclosure, the host is a human.

The anti-proliferative compounds (e.g., a 2,4-diaminoquinazoline compound such as a compound having a structure of Formula I, Formula Ia, Formula Ib, importazole, etc.) find use in a variety of applications. Applications of interest include, but are not limited to: therapeutic applications, research and manufacturing applications, and screening applications.

The methods are useful for treating a wide variety of cancers, including carcinomas, sarcomas, leukemias, and lymphomas. A patient having any cancer is suitable for treatment with a subject method.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

### Pharmaceutical Formulations, Routes of Administration, and Dosages

### Pharmaceutical Formulations

In some instances, as discussed above, a subject compound can be used to reduce the proliferation of cancer cells in vivo, e.g., an effective amount of a subject compound (e.g., a 2,4-diaminoquinazoline compound such as a compound having a structure of Formula I, Formula Ia, Formula Ib, importazole, etc.) is administered to an individual in need thereof. The subject compounds are also referred to herein as “agent” or “active agent.” For administration to an individual, a suitable compound (e.g., importazole or any of the other 2,4-diaminoquinazoline compound described herein) is formulated with one or more pharmaceutically acceptable excipients. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The term “unit dosage form,” as used herein, refers to physically discrete units suitable as unitary dosages for human and animal subjects, each unit containing a predetermined quantity of an active agent (e.g., a 2,4-diaminoquinazoline compound such as a compound having a structure of Formula I, Formula Ia, Formula Ib, importazole, etc.) calculated in an amount sufficient to produce the desired effect (e.g., reduced cancer cell proliferation) in association with a pharmaceutically acceptable diluent, carrier or vehicle. The specifications for an active agent depend on the particular compound employed and the effect to be achieved, and the pharmacodynamics associated with each compound in the host.

In the subject methods, a suitable active agent (e.g., the subject 2,4-diaminoquinazoline compounds such as a compound having a structure of Formula I, Formula Ia, Formula Ib, importazole, etc.) may be administered to the host using any convenient means capable of resulting in the desired outcome, e.g., reduction of disease, reduction of a symptom of a disease, etc. Thus, compounds of the present disclosure can be incorporated into a variety of formulations for therapeutic administration. More particularly, compound can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated, e.g., reduction of cancer cell proliferation to a desired level.

### Routes of Administration of the Subject Compounds

In pharmaceutical dosage forms, the subject compounds (e.g., a 2,4-diaminoquinazoline compound such as a compound having a structure of Formula I, Formula Ia, Formula Ib, importazole, etc.) may be administered in the form of their pharmaceutically acceptable salts, or they may also be used alone or in appropriate association, as well as in combination, with other pharmaceutically active compounds. The following methods and excipients are merely exemplary and are in no way limiting.

The agent can be administered to a host using any available conventional methods and routes suitable for delivery of conventional drugs, including systemic or localized routes. In general, routes of administration contemplated include but are not necessarily limited to, enteral, parenteral, or inhalational routes, such as intrapulmonary or intranasal delivery.

Conventional and pharmaceutically acceptable routes of administration include intranasal, intrapulmonary, intramuscular, intratracheal, subcutaneous, intradermal, topical application, intravenous, rectal, nasal, oral and other parenteral routes of administration. Routes of administration may be combined, if desired, or adjusted depending upon the agent and/or the desired effect. The composition can be administered in a single dose or in multiple doses.

For oral preparations, the subject compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. If oral administration is desired, the subject compounds may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Parenteral routes of administration other than inhalation administration include, but are not necessarily limited to, topical, transdermal, subcutaneous, intramuscular, intraorbital, intracapsular, intraspinal, intrasternal, and intravenous routes. Parenteral administration can be carried to effect systemic or local delivery of the agent. Where systemic delivery is desired, administration typically involves invasive or systemically absorbed topical or mucosal administration of pharmaceutical preparations. Where local delivery is desired, administration typically involves administering the composition to a desired target tissue, such a liver, heart, spine, etc. For local delivery, the administration may be by injection or by placement of the composition in the desired tissue or organ by surgery, for example.

Methods of administration of the agent through the skin or mucosa include, but are not necessarily limited to, topical application of a suitable pharmaceutical preparation, transdermal transmission, injection and epidermal administration. For transdermal transmission, absorption promoters or iontophoresis are suitable methods. Iontophoretic transmission may be accomplished using commercially available “patches” which deliver their product continuously via electric pulses through unbroken skin for periods of hours or several days or more.

The subject compounds of the present disclosure can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agent can also be delivered to the subject by enteral administration. Enteral routes of administration include, but are not necessarily limited to, oral and rectal (e.g., using a suppository) delivery. The subject compounds can be made into suppositories by mixing with a variety of bases such as emulsifying bases or water-soluble bases. A subject compound can be administered rectally via a suppository. The suppository can include vehicles such as cocoa butter, carbowaxes and polyethylene glycols, which melt at body temperature, yet are solidified at room temperature.

The subject compounds can be utilized in aerosol formulation to be administered via inhalation. An active agent can be formulated into pressurized acceptable propellants such as dichlorodifluoromethane, propane, nitrogen and the like.

### Dosages

Depending on the subject and condition being treated and on the administration route, an active agent (e.g., a 2,4-diaminoquinazoline compound such as a compound having a structure of Formula I, Formula Ia, Formula Ib, importazole, etc.) may be administered in dosages of, for example, 0.1 μg to 500 mg/kg body weight per day, e.g., from about 0.1 μg/kg body weight per day to about 1 μg/kg body weight per day, from about 1 μg/kg body weight per day to about 25 Kg/kg body weight per day, from about 25 μg/kg body weight per day to about 50 μg/kg body weight per day, from about 50 μg/kg body weight per day to about 100 μg/kg body weight per day, from about 100 μg/kg body weight per day to about 500 μg/kg body weight per day, from about 500 μg/kg body weight per day to about 1 mg/kg body weight per day, from about 1 mg/kg body weight per day to about 25 mg/kg body weight per day, from about 25 mg/kg body weight per day to about 50 mg/kg body weight per day, from about 50 mg/kg body weight per day to about 100 mg/kg body weight per day, from about 100 mg/kg body weight per day to about 250 mg/kg body weight per day, or from about 250 mg/kg body weight per day to about 500 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be about ten times the injection dose. Higher doses may be used for localized routes of delivery.

For example, a compound of the present disclosure (e.g., a 2,4-diaminoquinazoline compound such as a compound having a structure of Formula I, Formula Ia, Formula Ib, importazole, etc.) can be administered in an amount of from about 1 mg to about 1000 mg per dose, e.g., from about 1 mg to about 5 mg, from about 5 mg to about 10 mg, from about 10 mg to about 20 mg, from about 20 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 750 mg, or from about 750 mg to about 1000 mg per dose.

A typical dosage may be a solution suitable for intravenous administration; a tablet taken from one to six times daily, or one time release capsule or tablet taken once a day and containing a proportionally higher content of active ingredient, etc. The time release effect may be obtained by capsule materials that dissolve at different pH values, by capsules that release slowly by osmotic pressure, or by any other known means of controlled release.

Those of skill in the art will appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is one which provides up to about 1 μg to about 1,000 μg or about 10,000 μg of subject composition to reduce, e.g., cancer cell proliferation, in a subject animal.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present disclosure. Similarly, unit dosage forms for injection or intravenous administration may comprise the compound (s) in a composition as a solution in sterile water, normal saline or another pharmaceutically acceptable carrier.

### Combination Therapy

In some embodiments, a subject compound (e.g., the 2,4-diaminoquinazoline compounds of Formula I, Formula Ia, Formula Ib, importazole, etc.) is administered as an adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

In some cases, a subject compound (e.g., the 2,4-diaminoquinazoline compounds of Formula I, Formula Ia, Formula Ib, importazole, etc.) may be formulated with or otherwise administered in combination with other pharmaceutically active agents, including a second subject compound (e.g., of Formula I, Formula Ia, Formula Ib, importazole, etc.), other agents that reduce proliferation of cancer cells (e.g., chemotherapeutic agents), as well as sensitizers of agents that reduce proliferation of cancer cells, agents that reduce any side effects of the anti-proliferative agent(s), and the like.

In certain embodiments, a subject compound (e.g., a 2,4-diaminoquinazoline as described elsewhere herein) may be administered during the entire course of administration of a second compound. In one aspect, the subject compound may be administered for a period of time that is overlapping with the administration of the second compound, e.g. where administration of the subject compound begins before the administration of the second compound and the administration of the subject compound ends before the administration of the second compound ends. In still other embodiments, administration of the second compound begins before the administration of the subject compound and the administration of the second compound ends before the administration of the subject compound ends. Administration of the subject compound may begin before administration of the second compound begins and the administration of the second compound may end before the administration of the subject compound ends. Administration of the second compound may begin before administration of the subject compound begins and the administration of the subject compound may end before the administration of the second compound ends. As such, “in combination” can also refer to regimen involving administration of two or more compounds. “In combination with” as used herein also refers to administration of two or more compounds (e.g., a 2,4-diaminoquinazoline compound as described elsewhere herein, along with one or more additional active agents) which may be administered in the same or different formulations, by the same or different routes, and in the same or different dosage form type.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

“Paclitaxel” refers to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Identification of Importazole in a High-Throughput Screen

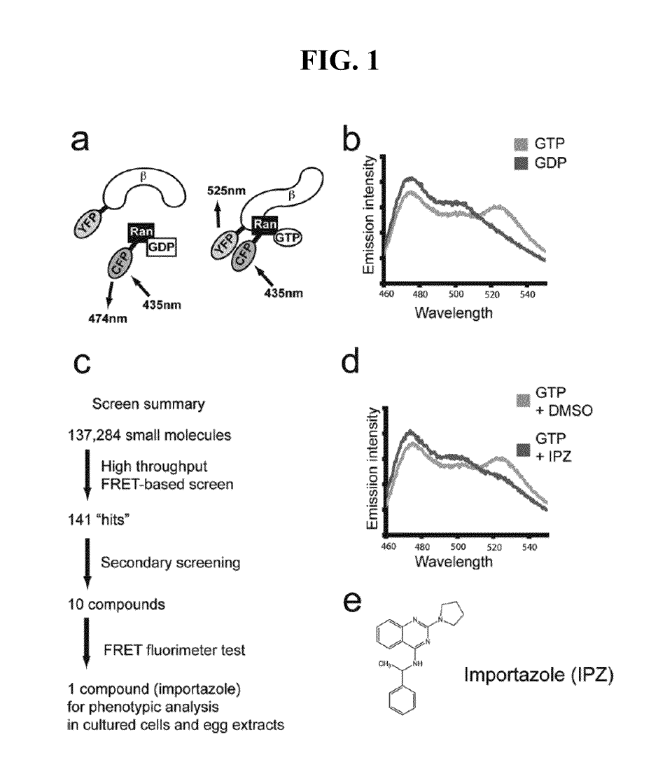

A reverse chemical genetic high-throughput screen (HTS) was performed to identify compounds that affect the interaction between RanGTP and importin-β using a fluorescence resonance energy transfer (FRET)-based assay with cyan fluorescent protein (CFP)-tagged Ran and yellow fluorescent protein (YFP)-tagged importin-β. These proteins bind one another only when CFP-Ran is guanosine triphosphate (GTP)-bound, which can be detected by changes in FRET (FIG. 1A and FIG. 1B). When CFP-Ran is incubated with RCC1, GTP, and YFP-importin-β, and the mixture is excited with 435 nm fluorescence in a fluorometer, a strong FRET signal is generated, as indicated by a decrease in the fluorescence intensity at 475 nm (the emission wavelength of CFP) and an increase in the fluorescence intensity at 525 nm (the emission wavelength of YFP). No FRET signal is generated if GDP is substituted for GTP, and nucleotide-specific interaction could also be observed biochemically, as S-tagged YFP-importin-β pulls CFP-Ran out of solution only in the presence of GTP. These results demonstrate that the FRET signal generated by CFP-RanGTP and YFP-importin-β is due to a physical interaction dependent upon the nucleotide state of Ran, and that the present approach could be used to identify compounds that interfere with the interaction between CFP-RanGTP and YFP-importin-β, resulting in a reduced FRET signal.

The assay was tested for suitability for high throughput screening (HTS) using a 384-well format and a fluorescence plate reader. FRET ratios (IFRET/ICFP) were calculated for each well and two commonly used statistical parameters were determined, the coefficient of variation (CV), which was 0.95% and 1.24% for reactions containing the GDP and GTP, respectively, and the Z′ value, which was 0.81, indicating that thesay was robust and appropriate for HTS. To facilitate rapid data analysis, software was developed to generate color-coded plate maps to identify compounds that reduced the FRET ratio by both an increase in CFP emission and a decrease in YFP emission, thereby eliminating compounds that altered the FRET ratio by contributing their own fluorescence at wavelengths in the range of our probes.

In total, 137,284 compounds were screened in duplicate (FIG. 1C), and 141 “hits” were selected for further analysis. Compounds that showed activity upon retesting in the original assay were analyzed in a unimolecular CFP and YFP FRET-based assay using a YIC sensor to confirm that the observed changes were not due to non-specific quenching or augmentation of either CFP or YFP emission. In a third assay, each compound was tested for its tendency to form aggregates that non-specifically inhibit β-lactamase. The 10 compounds that survived the secondary assays were obtained in larger quantities and tested again in the original CFP-Ran/YFP-importin-β FRET assay using a spectrofluorimeter. A 2,4-diaminoquinazoline designated by the inventors as “importazole”, reproducibly disrupted the FRET signal generated by CFP-Ran and YFP-importin-β and was analyzed further (FIG. 1D and FIG. 1E).

Materials and Methods

The high-throughput screen was carried out in collaboration with the Small Molecule Discovery Center (SMDC) at the University of California, San Francisco. Compounds were from ChemBridge, ChemDiv, SPECS, ChemRX, and Microsource. The complete content of this library can be found through the Small Molecule Discover Center website.

In the first step of the screen, compound dilution plates were made using a Multimek liquid handler and a Wellmate bulk dispenser by transferring 5 μl of compounds from stock plates into 384-well dilution plates (Corning, polypropylene, square wells). A bulk dispenser (Wellmate) was then used to transfer 45 μl of 2.77% dimethyl sulfoxide (DMSO) (diluted with reaction buffer). This yielded a compound concentration of 100 μM and a DMSO concentration of 12.5%. In the second step, the Multimek liquid handler was used to transfer 5 μl of solution from the dilution plates into the 384-well assay plates (Greiner, black, flat bottom). Next, the Wellmate bulk dispenser was used to transfer 20 μl of a solution containing CFP-Ran, RCC1, and GTP (diluted in reaction buffer) on top of the diluted compounds in the 384-well assay plates. This step yielded the following concentrations for each reaction component: CFP-Ran: 125 μM, RCC1: 40 nM, GTP: 400 μM, DMSO: 2.5%, and compound: 20 μM. The Wellmate bulk dispenser was then used to add 25 μl of diluted YFP-importin-β to each well in the assay plates. This step yielded the following final concentrations for each reaction component: CFP-Ran: 62.5 μM, YFP-importin-β: 62.5 μM, RCC1: 20 μM, GTP: 200 μM, DMSO: 1.25% and compound: 10 μM.

Each assay plate included 32 negative control wells (containing the GTP reaction+1.25% DMSO) and 32 positive control wells (containing the GDP reaction). These control wells were used to set the maximum and minimum fluorescence values for each plate individually. Each compound was tested in duplicate.

In the next step, the assay plates were loaded into the Analyst AD plate reader. Each well was excited with 435 nm fluorescence and emission was detected both at 475 nm (CFP) and 525 nm (YFP).

Because the high throughput screen generated a large amount of data, a software package using Perl was designed to analyze it. Text files generated by the Analyst AD included raw fluorescence values at 475 nm and 525 nm for each well in a 384-well plate. Each plate included 32 negative control wells (containing the GTP mixture+1.25% DMSO) and 32 positive control wells (containing the GDP mixture+1.25% DMSO). Data were processed by the software package in the following manner.

Positive control averages and standard deviations for the individual I_{CFP} and I_{FRET} emission values (475 nm and 525 nm respectively) were calculated using all 32 positive control wells. Similarly, the FRET ratio for each positive control well (I_{FRET}/I_{CFP}) was calculated and these values were used to generate an average I_{FRET}/I_{CFP} value and the standard deviation. The same calculations were performed using data from the negative control wells. Thus for each plate in the screen, the program calculated positive and negative control values that were used to set the maximum and minimum fluorescence intensities with which all other wells in the plate were compared. This permitted removal of many fluorescent compounds that interfered with CFP or YFP fluorescence indirectly causing excessively high or low emission readings.

In the next step, fluorescence values from each well in the plate were compared to both the average positive control value and the average negative control value and their corresponding standard deviations, which were used to make error bars. Wells were removed from further consideration if: (1) I_{FRET} was greater than that of the negative control average plus three standard deviations; (2) I_{FRET} was less than that of the positive control average plus three standard deviations; (3) I_{CFP} was greater than that of the negative control average plus three standard deviations; (4) I_{CFP} was less than that of the positive control average plus three standard deviations; (5) I_{FRET}/I_{CFP} was either less than the average I_{FRET}/I_{CFP} value plus three standard deviations for the positive control wells, or greater than the average I_{FRET}/I_{CFP} value plus three standard deviations for the negative control wells; or (6) I_{FRET}/I_{CFP} was not less than the average I_{FRET}/I_{CFP} value plus one standard deviation for the positive control wells (these compounds were considered to have no measurable effect on the interaction between CFP-Ran and importin-β because they did not affect I_{FRET}/I_{CFP}; some of these compounds were fluorescent (based on the emission intensities in the CFP and YFP channels) and were discarded as interfering compounds).

A compound was considered a “hit” and kept for further analysis if it satisfied all three of the following criteria: (1) it reproducibly (n=2) reduced I_{FRET}/I_{CFP} to a level in between the positive control average value minus two standard deviations and the negative control average value minus two standard deviations; (2) it reproducibly (n=2) reduced the I_{FRET} value to a level in between the negative control average value minus two standard deviations and the positive control average value minus two standard deviations; and (3) it reproducibly increased the I_{CFP} value to a level in between the positive control average value plus one standard deviation and the negative control average value plus one standard deviation.

The following reaction buffer was used for all FRET assays, including the high-throughput screen: 1× phosphate buffered saline (PBS), 5% glycerol, 2 mM MgCl₂, 1 mM dithiothreitol (DTT), 0.01% NP-40. For standard CFP-Ran/YFP-importin-β FRET assays, 50-100 nM CFP-Ran mixed with 20 nM RCC1 and 200 μM GDP or 200 μM GTP was immediately followed by addition of 50-100 nM YFP-importin-β. The reaction was excited using a Fluorolog 3 spectrofluorometer with 435 nm fluorescence and the emission was read between 460 nm and 550 nm. For the high-throughput screen, the concentrations of reaction components were as follows: CFP-Ran: 62.5 nM; YFP-importin-β: 62.5 nM; GTP or GDP: 200 μM; RCC1: 20 nM.

For the secondary screen, 141 hit compounds from the primary screen were tested for non-specific effects on fluorescence with FRET probe YIC that contains the importin-β-binding domain of importin-β flanked by CFP and YFP. When unbound in solution, this probe undergoes intramolecular FRET. In the second assay, the 141 hits were tested for nonspecific inhibition due to aggregation using a β-lactamase-based assay. Importazole was found to be soluble up to approximately 100 μM in water. Additionally, importazole was characterized by mass spectrometry and NMR to confirm its identity and purity.

For protein expression and purification, pET30a-derived constructs encoding importin-β with an N-terminal YFP fusion (pKW1532), a CFP-Ran fusion (pKW1543), and importin-β (pKW485) were transformed into BL21 cells (Invitrogen). Additionally, pQE32-derived Ran constructs (pKW356 [WT Ran], pKW 590 [RanQ69L]), a pQE9-derived Crm1 construct (pKW812), and a pQE60-derived transportin construct (pKW738) were transformed in to SG13 cells. All constructs were induced with IPTG at room temperature. Harvested cells were lysed using a French press. Fusion proteins were purified with Ni NTA resin using a standard protocol followed by gel filtration. RCC1 was purified as described in Azuma et al. (1996) J. Biochem. 120:82-91.

### Example 2

### Importazole Binds Importin-Beta In Vitro

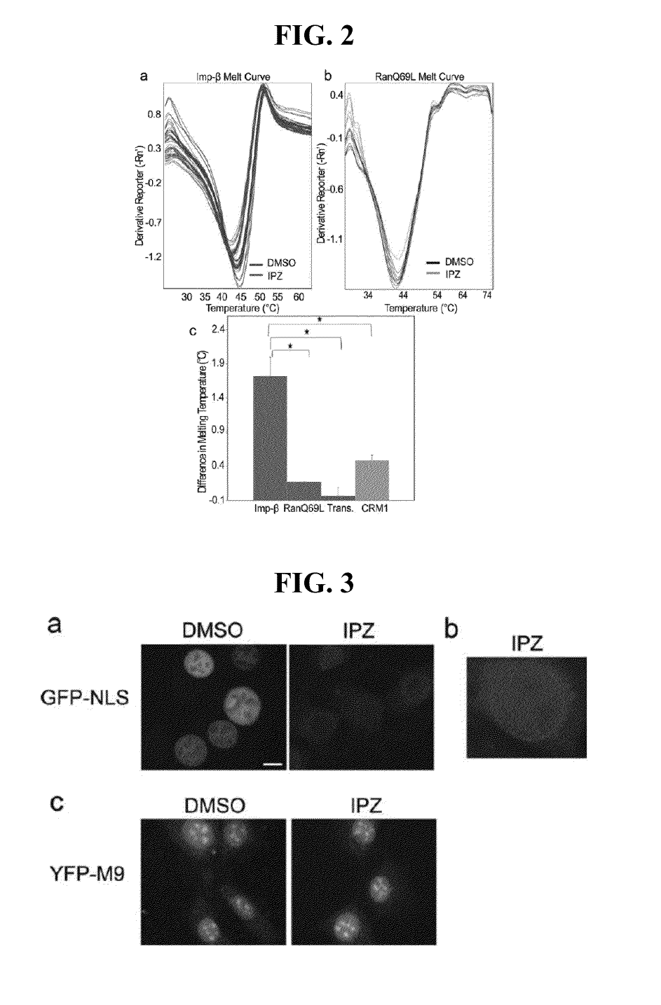

Although importazole blocked the FRET interaction between CFP-RanGTP and YFP-importin-β in vitro, it did not obviously affect the binding of the two proteins in pull-down assays. To begin elucidating the mechanism of importazole action, we tested whether importazole could alter the ability of importin-β to protect RanGTP from RanGAP-stimulated hydrolysis in vitro. Binding curves calculated from these data do not indicate that importazole disrupts the RanGTP/importin-β interaction, and if anything, suggest that importazole may slightly stabilize the complex. The inability of importazole to disrupt the RanGTP/importin-β interaction is not entirely surprising considering the multiple large interaction surfaces between the two proteins. One possible explanation for the importazole-induced FRET change is that importazole binding causes a conformational change that disrupts the CFP-RanGTP/YFP-importin-β FRET interaction without preventing binding. To test whether importazole binds to importin-β in vitro, a fluorescent thermal shift assay was carried out using the dye Sypro® Orange, since small molecule binding is expected to affect the thermal stability of a protein. Importazole reduced the melting temperature of importin-β by 1.72+/−0.27° C. (FIG. 2A and FIG. 2B), but was unaffected by a related compound of comparable hydrophobicity that did not interfere with CFP-RanGTP/YFP-importin-β FRET. In contrast, importazole did not significantly affect the melting curves of related importin-β family members transportin and CRM1, or that of RanGTP, suggesting that importazole binds preferentially to importin-β (FIG. 2C).

Materials and Methods

Experiments were performed using Applied Biosystems StepOnePlus™ Real-Time polymerase chain reaction (RT-PCR) System. Protein stocks were diluted in PBS and added 70% v/v to a Microamp® Fast 96-well Reaction Plate and maintained on ice. Compounds (importazole and control compound 3016) were then added at 30% v/v in 3% DMSO. Freshly prepared 100× water based-dilution of Sypro® Orange Protein Gel Stain was then added at 1% v/v to reach a final reaction volume of 20 μl. Samples were mixed by gentle pipetting. After sealing the plates with Microamp™ Optical Adhesive Film, the plate was subjected to a heating cycle composed of a 10 sec prewarming step at 25° C. and a gradient between 25° C. and 95° C. with a 0.3° C. ramp. Data was analyzed using the StepOnePlus™ Software v2.1.

For pulldown experiments to detect interaction between CFP-Ran and YFP-importin-β, all reactions were performed in buffer consisting of PBS+2 mM MgCl₂, 5% glycerol, 0.01% NP-40, and 1.0 mM DTT. Reaction buffer was combined with the following components in this order: CFP-Ran, RCC1, BSA, GDP or GTP, importazole or 100% DMSO, and YFP-importin-β, yielding the following final concentrations: CFP-Ran: 2.5 nM, RCC1: 20 nM, BSA: 0.1 mg/ml, GDP or GTP: 200 μM, importazole: 200 μM, YFP-importin-13: 5.0 nM. Reactions were incubated for 10 min at room temperature, 20.0 ml of S-protein agarose was added, and then incubated for an additional 30 min on a rotator before pelleting the agarose at 3,000 rpm for 1 min. The supernatant was removed followed by three washes with 500 ml reaction buffer. The S-protein pellet was resuspended in 15 μl of sodium dodecyl sulfate-polyacrylamide gel electrophoresis (SDS PAGE) sample buffer and boiled briefly. After spinning down the S-protein pellet, 10 μl of the sample was analyzed by SDS PAGE.

For RanGTP protection assays, all steps were performed in buffer containing 50 mM HEPES pH 7.6 with 2.5 mM MgCl₂. Loading the Ran with GTP: 40 nM Biotin labeled RCC1 was bound to streptavidin agarose resin for 30 min rotating at 4° C. The Biotin labeled RCC1 was a gift of D. Halpin. The beads were washed with fresh buffer to remove any free RCC1, and 16 μM nucleotide free Ran and 51 μM GTPγP³² were added and Ran was allowed to load for 30 min at room temperature. The beads were then removed using a spin column, and the remaining supernatant was filtered through a Sephadex G-50 column to remove free GTPγP³². The loaded Ran was then diluted to 1.6 μM for further use. Performing the assay: All reactions were performed in a final volume of 200 μL at and in buffer containing 50 mM HEPES pH 7.6 with 2.5 mM MgCl₂. Importin-β was pre-incubated with either 0.5% DMSO or 50 μM importazole in 0.5% DMSO and 100 nM RanGTPγP³² at room temperature. 1 μM RanGAP was added to start each reaction, and the reaction was allowed to proceed for 5 min before the reaction was stopped using 1 ml of a solution containing 7% charcoal, 10% ethanol, 0.1 M HCl, and 10 mM KH₂PO₄. The resulting solution was spun at 10,000 RPM for 2 min in a tabletop centrifuge to pellet the charcoal, and the resulting supernatant was removed and counted for three min per sample in a liquid scintillation counter.

### Example 3

### Importazole Disrupts Importin-Beta/RanGTP-Mediated Nuclear Import

Because importazole binds importin-β and affects the RanGTP/importin-β interaction, importazole was tested to determine whether it inhibits the nuclear import of any protein bearing a classical nuclear localization signal (NLS). This was first tested using permeabilized HeLa cells, in which nuclear import of a GFP-NLS reporter can be reconstituted in vitro. Digitonin-permeabilized cells were incubated with a GFP-NLS reporter plus Xenopus laevis egg extracts as a source of soluble transport factors including Ran, importin-α, and importin-β. Whereas rapid nuclear accumulation of GFP-NLS occurred in the presence of the solvent DMSO, importazole blocked import and the reporter became enriched at the nuclear envelope, where RanGTP functions to induce cargo release from importin-β (FIG. 3A and FIG. 3B). In contrast, importazole did not block nuclear import mediated by transportin, an importin-β family member that utilizes the M9 import signal together with RanGTP to import hnRNP proteins (FIG. 3C).

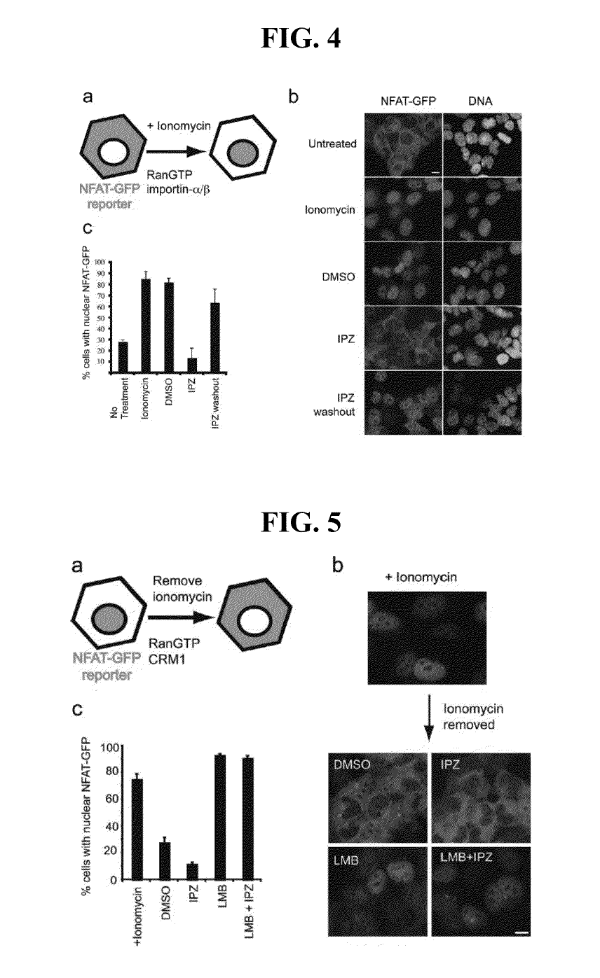

To investigate whether importazole is cell permeable and active in living human cells, a cell line was generated that stably expresses a GFP-tagged version of the transcription factor NFAT (nuclear factor of activated T cells), which shuttles between the nucleus and the cytoplasm in a calcium-regulated manner and is imported by importin-α/β and exported by CRM1. At steady state NFAT is predominantly cytoplasmic. An increase in cytoplasmic calcium induced by the ionophore ionomycin leads to the accumulation of NFAT in the nucleus (FIG. 4A). NFAT import can be reverted upon ionophore withdrawal, (FIG. 5A) providing an inducible system ideal for testing the effects of importazole on importin-β-mediated nuclear import and CRM1-mediated nuclear export, both of which are dependent upon RanGTP.

Cells were pretreated with 40 μM importazole for 1 hour followed by 30 minutes of ionomycin treatment in the continued presence of importazole. Whereas control cells treated with DMSO or the control compound 3016 displayed a robust nuclear accumulation of the NFAT-GFP reporter after ionomycin addition, there was virtually no import of NFAT-GFP in importazole treated cells (FIG. 4B, quantified in FIG. 4C). Importazole displayed an IC₅₀ of approximately 15 μM for inhibition of NFAT-GFP import. This effect was reversible upon importazole washout, which restored ionomycin-induced import of NFAT-GFP to near control levels (FIG. 4B and FIG. 4C). Thus, it is possible to use importazole in drug-washout experiments to study the Ran/importin-β pathway in cells. The reversibility of importazole required 1 hour of recovery time between washing out the drug and adding ionomycin, and did not require new protein synthesis.

To further assess the specificity of importazole, its effects on CRM1-mediated export of NFAT-GFP were tested. Export of NFAT-GFP occurred efficiently in the presence or absence of importazole, but was blocked by leptomycin B, a specific CRM1 inhibitor (FIG. 5B, quantified in FIG. 5C). Importantly, when cells were treated with both leptomycin B and importazole upon ionomycin washout, NFAT-GFP was still restricted to the nucleus (FIG. 5B and FIG. 5C), confirming that importazole treatment does not non-specifically damage the nuclear envelope allowing proteins to leak out into the cytoplasm. Consistent with the concentration of importazole sufficient to impair nuclear import, it was found that importazole has an IC₅₀ of approximately 22.5 μM in HeLa cells following treatment over a 24-hour period.

Taken together, the nuclear import experiments indicate that importazole is specific for importin-β-mediated protein import. No effect on transportin-mediated import or CRM1-mediated export was detected. Furthermore, these results indicate that importazole does not impair RCC1-dependent loading of Ran with GTP or the function of RanGTP itself since the export function of CRM1 critically depends on the formation and function of RanGTP.

Materials and Methods

A GFP-NFAT expression plasmid (pKW520) was generated by inserting a BamH I/Hind III-cleaved NFATC1 cDNA fragment into pEGFP-C1 (Clontech) digested with Bgl II and Hind III. The construct was stably transfected into HEK 293 cells and a single clone expressing moderate levels of NFAT-GFP was selected and maintained in Opti-mem media (Gibco) plus 4% fetal bovine serum, 1% penicillin/streptomycin, and 200 μg/ml G418. HeLa cells were grown and maintained according to standard protocols.

HeLa cells were permeabilized and treated with an import reporter and cytosol from Xenopus laevis oocytes as described by Adam et al. (1990) J. Cell Biol. 111:807-816.

For all import and export experiments, HEK 293 cells stably expressing NFAT-GFP were grown on glass coverslips to approximately 50% confluency prior to drug treatment. In all cases, importazole was used at 40 μM and leptomycin B was used at 10 ng/ml. For controls, DMSO was used at a concentration of 0.4%. Ionomycin was added at 1.25 μM. Importazole and leptomycin B treatments were all for 1 hour. In all experiments cells were fixed with 4% formaldehyde prior to fluorescence microscopy. DNA was visualized with 1 μg/ml Hoechst dye. For quantification, 100 cells from each condition were analyzed and the percentage that showed nuclear accumulation of NFAT-GFP calculated.

For HeLa cell viability assays to obtain an IC₅₀, 10,000 actively growing HeLa cells per well were transferred to opaque white 96 well tissue culture plates at a final volume of 100 μl per well in DMEM plus 4% fetal bovine serum and 1% penicillin/streptomycin. Cells were allowed to grow at 37° C. for 24 hours. Individual wells were then treated for 12 hours with one of the following conditions: 1% DMSO, 5, 10, 20, 30, 40, 50, 60, 75, 100 μM IPZ. Following this treatment, the media was replaced and cells were treated for another 12 hours under the same conditions. The plates were then allowed to equilibrate to room temperature for 30 min, after which 100 μl of room temperature CellTiter-Glo reagent from the Promega CellTiter-Glo Luminescent Cell Viability Assay kit was added to each well. Plates were shaken for 2 min, incubated at room temperature for 10 min, then read on a luminometer. The average background signal for the plate was subtracted from the value of each individual well, and the resulting values were normalized to the signal level of the DMSO containing well.

### Example 4

### Importazole Blocks Spindle Assembly in Xenopus Egg Extracts, but does not Affect Pure Microtubules

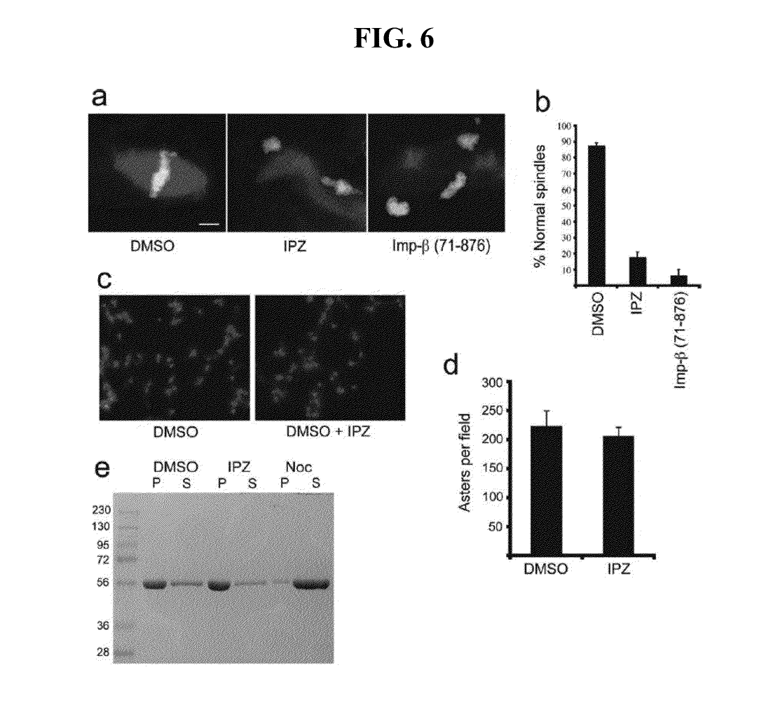

In order to determine whether importazole also disrupts mitosis, importazole was first tested in metaphase-arrested Xenopus egg extracts, which rely heavily on a RanGTP gradient for spindle assembly around sperm chromosomes. Addition of 100 μM importazole, but not the solvent DMSO, strongly inhibited spindle assembly, preventing normal bipolar microtubule structures from forming around 80% of sperm nuclei (FIG. 6A and FIG. 6B). The effect was similar to that of adding a truncated importin-β (amino acids 71-876), a version that no longer binds to RanGTP and therefore sequesters its cargoes. Although importazole significantly weakened spindle microtubule density, it was not a general microtubule inhibitor, since it did not impair the formation of microtubule asters in the extract induced by the microtubule stabilizing agent DMSO (FIG. 6C and FIG. 6D) or affect the polymerization of pure microtubules, in contrast to nocodazole (FIG. 6E). Thus, importazole caused dramatic effects on spindle assembly consistent with the known role of the importin-β/RanGTP pathway in the Xenopus egg extract system, and is not a general microtubule inhibitor.

Materials and Methods

Xenopus laevis egg extracts were prepared as described by Hannak et al. (2006) Nat. Protoc. 1:2305-2314. For in vitro spindle assembly, Xenopus laevis sperm DNA was added to egg extracts supplemented with rhodamine-labeled tubulin. Asters were formed by addition of 5% DMSO. DNA was stained with Hoechst dye. The formation of microtubule-based structures was assessed using epifluorescence microscopy after a 30-minute room temperature incubation. In vitro microtubule polymerization and pelleting assays were performed by incubating 25 μM bovine tubulin, 1 mM GTP, and 5% DMSO in BRB80 buffer (80 mM PIPES, 1 mM MgCl₂, 1 mM EGTA, pH 6.8) at 37° C. for 30 minutes. Polymerized microtubules were pelleted through a sucrose cushion, resuspended, and analyzed by SDS PAGE.

### Example 5

### Importazole Impairs Mitotic Cargo Release and Reveals Novel Functions for the Importin-Beta/RanGTP Pathway in Human Cells

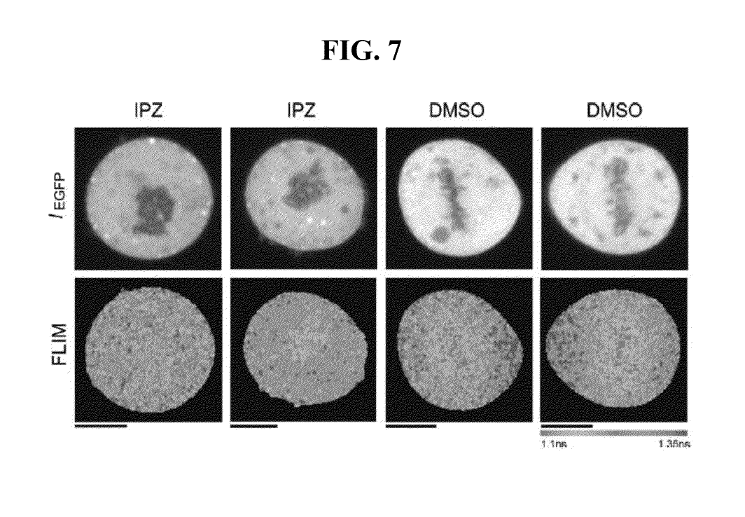

A key feature of importazole, a cell-permeable importin-β/RanGTP inhibitor, is its ability to dissect novel roles of this pathway in dividing human cells, which also provide a system to analyze mitotic gradients of released cargos using FRET probes. If importazole disrupts the interaction of importin-β with RanGTP, then the chromatin-localized FRET gradient of the cargo probe Rango should be reduced, since it undergoes FRET when released from importin-β in HeLa cells. As predicted, the difference in fluorescence lifetime of the donor GFP of the Rango-3 probe between the chromosomes and distal cytoplasm was significantly reduced in the presence of importazole compared to controls, from an average of 0.12+/−0.4 ns to 0.07+/−0.03 ns due to reduced FRET (FIG. 7, p-value: 1.3×10⁻⁷). Thus, importazole impairs mitotic importin-β cargo release in HeLa cells.

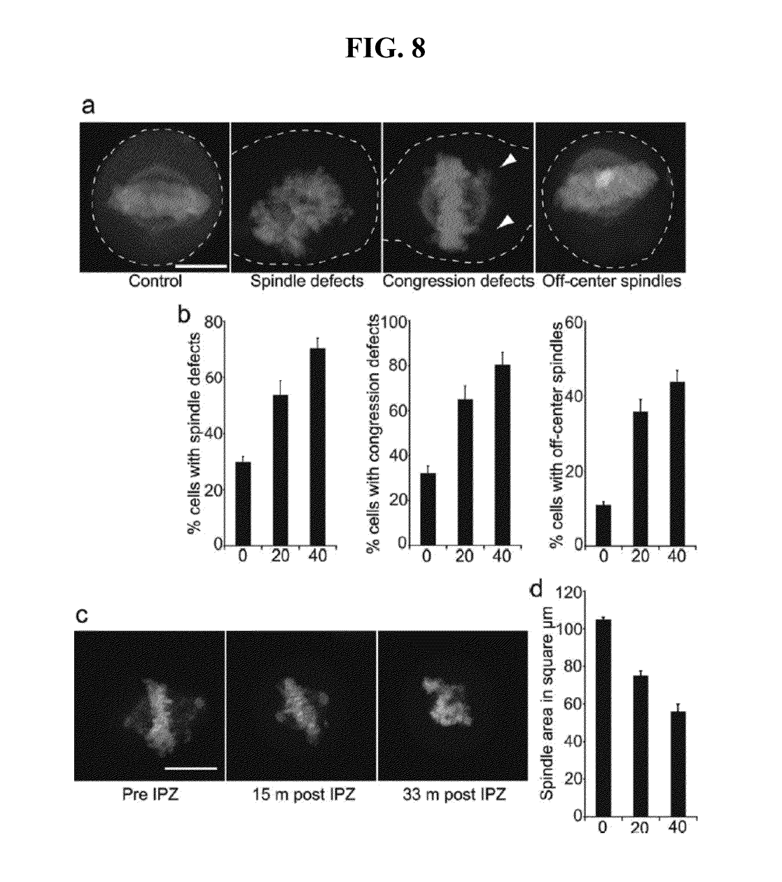

To examine the consequences of importazole on mitosis, HeLa cells treated for one hour were fixed and stained for tubulin and chromosomes. Control metaphase figures displayed robust spindles with a mean area of 105 μm², and were centrally located within the cell with chromosomes aligned on the metaphase plate (FIG. 7A and FIG. 7D). Importazole treatment caused dose-dependent defects in spindle assembly, chromosome alignment, and spindle size (FIG. 8). Interestingly, importazole also led to spindle positioning defects, with more than 40% of the cells displaying off-center spindles (FIG. 8A and FIG. 8B). Spindle positioning was not previously attributed to the Ran pathway and this phenotype may be a consequence of astral microtubule disruption by importazole. Previous studies have most likely not revealed this role of the Ran pathway in mitosis because they were performed in cell free systems such as Xenopus extract where spindle positioning could not be assessed. The discovery of this spindle misalignment phenotype demonstrates the importance of importazole as a tool to study the Ran pathway in mitosis.

Materials and Methods

The Rango-3 FRET sensor is an improved version of Rango and was created by replacing Cerulean-EYFP donor-acceptor pair in Rango with EGFP as a donor and non-fluorescent acceptor sREACh which was modified by the introduction of mild dimerization mutations. Time-correlated single photon counting (TCSPC) datasets were acquired with a Plan-Apochromat 63×/1.40 NA oil immersion lens on an inverted Zeiss LSM710 NLO microscope equipped with a Becker & Hickl SPC-830 TCSPC controller. Samples were excited by one-photon 485 nm pulses generated by a frequency doubling 970 nm 80 MHz Ti:Sapphire laser (Coherent MiraSHG). The emission was collected from a custom side port, filtered through a 525 nm bandpass filter (ET525/50 Chroma) and detected by a HPM-100-40 module (Becker & Hickl) containing a hybrid Hamamatsu R10467-40 GaAsP photomultiplier. Two to three days before the experiment, HeLa cells were transfected with a pSG8 plasmid containing the Rango-3 open reading frame (pK135) to induce sensor expression. Treatment with importazole or DMSO was started one hour before imaging and continued for up to one hour in an environmental chamber built on the microscope (37° C., 5% CO₂). Recording conditions were chosen to limit emission to approx 1−2×10⁶ counts per second, and images of 128×128 pixels (1024 time bins/pixel) were averaged over 60 seconds. Fluorescence lifetime images were produced and analyzed using SPCI software (Becker & Hickl).

Cells were fixed in 4% formaldehyde and 0.1% glutaraldehyde in PHEM (60 mM PIPES, 25 mM HEPES, 10 mM EGTA, 2 mM MgSO₄) at 37° C. for 15 minutes followed by permeabilization with 0.1% triton X-100 for 2 minutes at room temperature. Cells were then washed and blocked (PHEM+5% FBS+0.2% saponin) and stained by standard techniques using the E7-A anti β tubulin antibody (Developmental Studies Hybridoma Bank) diluted 1:1000 and Hoechst dye.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of reducing proliferation of a cancer cell, the method comprising contacting the cancer cell with a compound of the formula: 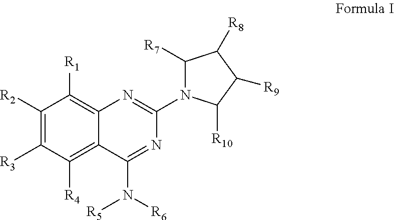
wherein R₁, R₂, R₃, R₄, R₅, R₆, R₇, R₈, R₉ and R₁₀ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

2. The method of claim 1, the method comprising contacting the cancer cell with a compound of the formula: 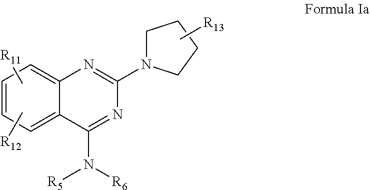
wherein R₅ and R₆ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
wherein R₁₁, R₁₂ and R₁₃ are each attached to their respective rings at any available position on the ring and are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

3. The method of claim 1, the method comprising contacting the cancer cell with a compound of the formula: 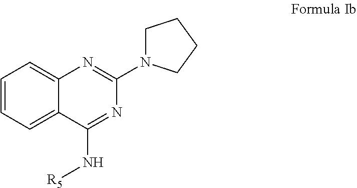
wherein R₅ is selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

4. The method of claim 1, wherein R₅ is a substituted or unsubstituted alkyl group.

5. The method of claim 4, wherein R₅ is: 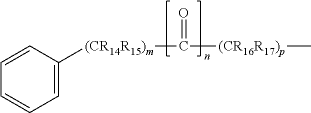
wherein R₁₄, R₁₅, R₁₆ and R₁₇ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
m, n and p are each independently an integer from 0 to 6.

6. The method of claim 4, wherein R₅ is: 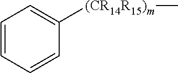
wherein R₁₄ and R₁₅ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
m is an integer from 1 to 6.

7. The method of claim 1, the method comprising contacting the cancer cell with a compound of the formula:

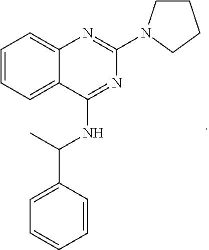 [SMILES: CC(Nc1nc(N2CCCC2)nc2ccccc12)c1ccccc1]

8. A method of modulating importin-beta-mediated nuclear import in a eukaryotic cell, the method comprising contacting the eukaryotic cell with a compound of the formula: 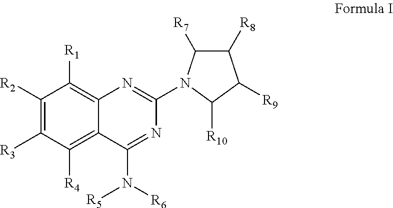
wherein R₁, R₂, R₃, R₄, R₅, R₆, R₇, R₈, R₉ and R₁₀ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

9. The method of claim 8, the method comprising contacting the eukaryotic cell with a compound of the formula: 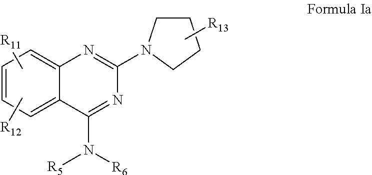
wherein R₅ and R₆ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
wherein R₁₁, R₁₂ and R₁₃ are each attached to their respective rings at any available position on the ring and are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

10. The method of claim 8, the method comprising contacting the eukaryotic cell with a compound of the formula: 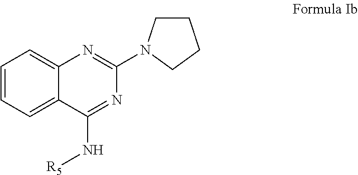
wherein R₅ is selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

11. The method of claim 8, wherein R5 is a substituted or unsubstituted alkyl group.

12. The method of claim 11, wherein R₅ is: 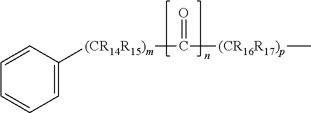
wherein R₁₄, R₁₅, R₁₆ and R₁₇ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
m, n and p are each independently an integer from 0 to 6.

13. The method of claim 11, wherein R₅ is: 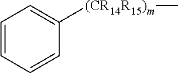
wherein R₁₄ and R₁₅ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
m is an integer from 1 to 6.

14. The method of claim 8, the method comprising contacting the eukaryotic cell with a compound of the formula:

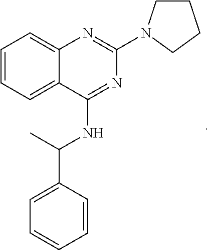 [SMILES: CC(Nc1nc(N2CCCC2)nc2ccccc12)c1ccccc1]

15. A pharmaceutical formulation comprising:
a compound of the formula: 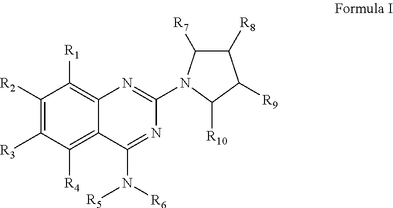
wherein R₁, R₂, R₃, R₄, R₅, R₆, R₇, R₈, R₉ and R₁₀ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; or a pro-drug, a pharmaceutically acceptable salt, an analog, or a derivative thereof; and
a pharmaceutically acceptable excipient.

16. The pharmaceutical formulation of claim 15, wherein the compound has the formula: 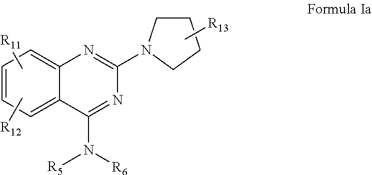
wherein R₅ and R₆ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
wherein R₁₁, R₁₂ and R₁₃ are each attached to their respective rings at any available position on the ring and are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

17. The pharmaceutical formulation of claim 15, wherein the compound has the formula: 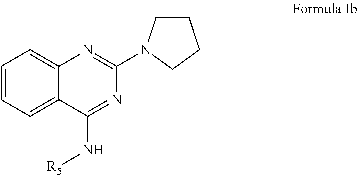
wherein R₅ is selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group;
or a pro-drug, a pharmaceutically acceptable salt, an analog, or a derivative thereof.

18. The pharmaceutical formulation of claim 15, wherein R₅ is a substituted or unsubstituted alkyl group.

19. The pharmaceutical formulation of claim 18, wherein R₅ is: 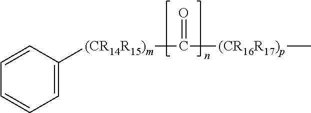
wherein R₁₄, R₁₅, R₁₆ and R₁₇ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
m, n and p are each independently an integer from 0 to 6.

20. The pharmaceutical formulation of claim 18, wherein R₅ is: 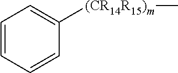
wherein R₁₄ and R₁₅ are each independently selected from H, a halogen, a substituted or unsubstituted alkyl group, a substituted or unsubstituted alkenyl group, a substituted or unsubstituted alkynyl group, a substituted or unsubstituted cyclic group, a substituted or unsubstituted heterocyclic group, a substituted or unsubstituted aryl group, a substituted or unsubstituted heteroaryl group, an ether, a substituted or unsubstituted amine, an ester, and an amide group; and
m is an integer from 1 to 6.

21. The pharmaceutical formulation of claim 15, wherein the compound has the formula: 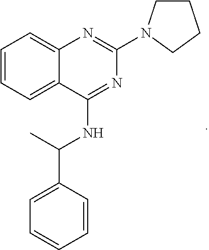 [SMILES: CC(Nc1nc(N2CCCC2)nc2ccccc12)c1ccccc1]
